(19) United States
(12) Patent Application Publication
(10) Pub. No.: US 2019/0192742 A1
(43) Pub. Date: Jun. 27, 2019

(54) FIBER-REINFORCED BIORESORBABLE IMPLANT AND METHOD FOR PRODUCING SAME

(71) Applicant: Karl Leibinger Medizintechnik GmbH & Co. KG, Mühlheim (DE)

(72) Inventors: Adem AKSU, Mühlheim (DE); Frank REINAUER, Mühlheim (DE); Tobias WOLFRAM, Mühlheim (DE); Lorenz GABELE, Mühlheim (DE)

(21) Appl. No.: 16/329,626

(22) PCT Filed: Nov. 22, 2017
(86) PCT No.: PCT/EP2017/071129

§ 371 Date: Feb. 28, 2019

(30) Foreign Application Priority Data
Sep. 1, 2016 (DE) 10 2016 116 387.2

(51) Int. Cl. A61L 27/58; A61L 27/48

(52) U.S. Cl. CPC A61L 27/58; A61L 27/48

(57) ABSTRACT

The invention relates to a bioresorbable implant (1) for supplementing or replacing hard tissue and/or soft tissue, comprising at least one reinforcing fiber/fiber bundle or a fiber structure or fiber construct (2) which is made of a first material component and which is embedded into a matrix (3) after being mixed with a second material component. The material of the first material component contains at least one of the elements of the group consisting of silk, chitosan, collagen, polycaprolactone, poly(D,L-lactide), poly(lactide-co-glycolide), polyglycolide, polyurethane and polypropylene, wherein the second material component is present in granular or powdery form at the point in time at which the material component is mixed with the fibers/fiber bundle, fiber structure or fiber construct (2). The invention likewise relates to a method for producing such implant (1).

The invention relates to a bioresorbable implant for supplementing or replacing hard tissue and/or soft tissue such as cartilage, bone or any other tissue. The bioresorbable material includes at least one reinforcing fiber. Said at least one reinforcing fiber preferably is in the form of a reinforcing fiber bundle/a reinforcing fiber structure or a reinforcing fiber construct. The at least one reinforcing fiber is made from a first material component and is embedded into a matrix after being mixed with a second material component. The material of the first material component contains at least one or a combination of the elements of the group consisting of silk, chitosan, collagen, polycaprolactone, poly(D,L-lactide), poly(lactide-co-glycolide), polyglycolide, polyurethane and polypropylene.

It is emphasized once again that also fiber bundles and fiber structures as well as fiber constructs are understood by fibers herein.

Equally, the invention relates to a method for producing a bioresorbable material.

On the one hand, from prior art bioresorbable implants are known which are made from only one raw material. In order to bring about an improvement of the biological reactions to the degradation of the resorbable materials, on the other hand technologies which are based on plural materials have prevailed in the field of implants. However, the improved biological reactions are confronted with a loss of mechanical strength, which under certain circumstances may result in early failure of the implant under mechanical load. For example, such implants which are used to regenerate defects usually have no sufficient mechanical stability to replace the tissue to be replaced to the full extent directly after they have been inserted by operation.

A generic implant is known from the German patent application 10 2010 034 471 A1. It discloses an implant comprising a filament which includes an elongate preferably braided filament body and a coating at least partially surrounding the filament body. The filament of this patent application is made from polyethylene and/or polypropylene, while the coating consists of a resorbable material and, where necessary, of additives.

From the European patent document 1 537 883 B1, too, an implant is known. The latter is aimed at repairing tissue injury and defects and has a biocompatible structure comprising reinforcing material.

In the European patent application 2 081 020 A2 a tissue implant is disclosed which is made from bioresorbable components and/or from non-bioresorbable components. As non-resorbable component natural or synthetic silk is considered, for example.

The US patent application with the serial number 2004/0054372 A1 is directed to biologically degradable composite materials for use as an implant. Said implant comprises a biodegradable fiber-reinforced composite, a matrix as well as fibers.

It is a drawback of the a.m. prior art that by adaptation of the implant geometries to individual characteristics the amount of material used is increased. Apart from the decreasing cost-effectiveness due to the increased use of material, the biological processes are caused to be deteriorated which negatively affects clinical results. For, as is known, concentration-related negative clinical reactions can be caused by an increased amount of substances to be degraded from the implant.

Equally, in prior art the composite materials to be joined are produced primarily by means of a so-called prepreg technology (abbreviated for “pre-impregnated fibers”). The latter are textile fiber-matrix semi-finished products pre-impregnated with reaction resins which are hardened under temperature and pressure for producing implants. This is cost-intensive and time-consuming.

Consequently, it is the object of the invention to eliminate or at least to alleviate the drawbacks known from the prior art and, especially, to make available an implant which, while using as little material as possible, enables quick bioresorbable characteristics as well as an efficient production.

According to the invention, this is ensured by the fact that the second material component is present in granular or powdery form at the time of mixing with the at least one reinforcing fiber/the fiber bundle/the fiber structure or fiber construct. Thus, the first material component is present in the form of a fiber/fiber bundle or in the form of a fiber structure or fiber construct and the second material component is present, for example, in the form of powder or granules which have to be mixed with each other in different quantities and material compositions. In this way, high flexibility of the characteristics to be achieved is ensured as the mixing of at least one fiber/one fiber bundle/one fiber structure or fiber construct and of a powder or granules can be individually designed while ensuring high reliability.

Advantageous embodiments are the subject matter of the subclaims and shall be illustrated in detail hereinafter.

Preferably, alternatively or additionally the second material component may also be present in liquid form. The at least one fiber can be aligned in a liquid second material component in a highly flexible manner. Further, a liquid second material component enables different densities and strengths to be realized.

It is advantageous when the matrix is made from the second material component and at least one further material component. This causes the characteristics of the matrix, such as regarding the strength or regarding the degradation kinetics or the biological adaptation, to be specifically influenced by adding a further component.

As soon as the second material component comprises ceramic phosphate-based components, high strength of the matrix which adopts a structural function is ensured. This increases the reliability of the implant and the biological compatibility thereof.

In a preferred embodiment, the entirety of fibers contains at least two of the elements of the group consisting of silk, chitosan, collagen, polycaprolactone, poly(D,L-lactide), poly(lactide-co-glycolide), polyglycolide, polyurethane and polypropylene. It is possible here that both one fiber is made from plural materials and that individual fibers include the same material but among each other include different materials. Each individual one of the elements from the afore-mentioned group offers its individual advantages which are known from material science. Thus, the selection of which of the elements is/are to be selected is dependent on the respective general conditions. In this context, the strength, the biological compatibility, the cost-effectiveness as well as the ratio of volume and mass of the fiber component to the matrix are listed as influencing factors.

Another advantage is offered when the fiber has at least one elevation and/or recess to increase an interacting surface between the matrix and the fiber. This allows for a robust seat of the fiber within the matrix. In this way, the implant withstands the load even in the case of unexpectedly high external and internal force impacts.

In an advantageous embodiment, plural solid particles allowing to control a resorption time are arranged in the matrix, wherein the solid particles have a mass percentage of 5% to 25% as measured by the mass of the bioresorbable material/implant. The higher the mass percentage of the solid particles within the matrix, the higher the influence of time exerted by them. In this context, it has to be evaluated how many percent by weight can be used while observing the general conditions of strength and while considering the biological characteristics. As the second material component is in the form of powder and/or granules at the time of being mixed, the admixture of the solid particles can be realized without great additional effort when manufacturing the implant.

Even when mixing of the individual material components is possible by generative, subtractive and 3D-shaping manufacture, precise and reliable production of the bioresorbable material is promoted. The generative layering method is promoted by the powdery/granular form and the filament shape. The generative fabrication causes support structures as they are required in different methods of rapid prototyping to be omitted, which, inter alia, has a positive effect on the amount of material to be used.

This is analogously applicable to further 3D-shaping methods, such as e.g. compression molding. As a further option for mixing the individual components LCM (“lithographic-based ceramic manufacturing”) or electro-spinning offers itself.

Furthermore, advantages will be apparent when the particles of the second material component granular or powdery at the time of mixing adopt an integral and merging shape after a predetermined time window. In this way, any powder form of the second material component is omitted with appropriate post processing, which has a favorable effect on force wear within the implant as no more phase limits within the material are present.

In another preferred embodiment, the particles of the second material component granular or powdery at the time of mixing contain at least one of the elements of the group consisting of magnesium, calcium, hydroxyapatite, alpha- and/or beta-tricalcium phosphate and lime to specifically influence the degradation behavior of the bioresorbable implant. In this way, the bioresorption of the implant can be variably adapted depending on a patient's state of health so as to guarantee quick healing without any complications.

A method for producing a bioresorbable implant is likewise part of the invention. Said method includes various steps which are preferably carried out successively in time. Providing the fiber from the first material component will be followed by providing granular or powdery particles or particles in a liquid state of the second material component. When both material components are brought to a state, as regards the external conditions such as arrangement or temperature thereof, in which they are prepared for being mixed, said mixing takes place to obtain the bioresorbable implant from the first material component and the second material component. Said implant has a three-dimensional geometry and may subsequently be finished, where necessary, in order to be configured as to its shape and/or surface texture to be complementary to the hard tissue subject to replacement.

The method according to the invention is optionally extended by a step of admixing a further component in order to optimize the implant as regards its degradation kinetics as well as its biological interaction with the patient's body.

It is equally part of the invention that the second material component surrounds the fiber of the first material component such that a three-dimensions expansion and geometry of the implant is defined. Said expansion/geometry can be variably designed and thus can be optimally adapted to the varying conditions.

In accordance with the invention, the fiber proportion in mass percentage of the implant ranges from 5% to 95%. Especially preferred are configurations in which the mass percentage is 5%, 15%, 20%, 30 to 55% or 60 to 95%.

As the density of the different material components is not constant, the fiber proportion in volume percentage does not necessarily correspond to that in mass percentage. In volume percentage the fiber proportion ranges from 5% to 95%. Especially preferred are configurations in which the volume percentage is 5%, 15%, 20%, 30 to 55% or 60 to 95%.

The fibers are preferably arranged so that they optimize the strength characteristics of the implant. In addition, it is possible to provide the bioresorbable fiber-reinforced implant with further materials. They are advantageously contained in particulate form. Examples of said particles are magnesium, iron, barium, strontium, calcium, hydroxyapatite, alpha- and/or beta-tricalcium phosphate and lime. It is possible that all particles are made from the same element/material or that the individual particles are different. The specific application, i.e. the situation of the patient, will decide on whether and which particles will be utilized. Each of said secondary materials is utilized in such way that it has a supporting effect on the natural bone formation.

Of advantage, the proportion of said particles in the total mass of the implant ranges from 5% to 25%, and is approximately 10%, 15% or 20%.

As regards its volume, the proportion of said particles in the total mass is advantageously dimensioned to be from 5% to 25%, approximately 10%, 15% or 20%.

The strength of the implant including particles depends on the geometries which the particles exhibit. Also, the degradation kinetics are influenced by that. Preferably, the particles are substantially spherical. Accordingly, ball diameters of from 30 μm to 60 μm are common. Equally, definitely smaller ball diameters of from 30 nm to 60 nm can be used according to the invention.

In an advantageous embodiment, the particle diameter ranges from 1 μm to 10 μm, further preferred from 15 μm to 25 μm and even further preferred from 50 μm to 150 μm. The respective application, viz. the situation of the patient, decides on the particle size which will be used.

As regards the at least one fiber, also the geometry can be varied. Of preference, the fibers have a length of from 1 mm to 10 mm. In larger implants the fiber length ranges from 50 mm to 100 mm.

The fiber in one embodiment has a circular cross-section. The latter has a diameter of about 30 μm. Likewise, a fiber diameter of from 10 nm to 1 μm is possible. In another example configuration, the dimension of the fiber diameter ranges from 5 μm to 15 μm and in even another configuration the dimension ranges from 100 μm to 500 μm.

The entirety of the fibers is preferably composed to form a fiber construct which is orientated e.g. net-like relative to each other. Said structure can be configured in a plane or also three-dimensionally. The fiber construct has an orientation of individual partial fibers which are interwoven in a net-like manner.

Of preference, the fibers have a structural surface topography to intensify/to enlarge the interacting surface between the individual fibers and the matrix. This increases the mechanical stability/robustness/strength of the implant according to the invention.

The chitosan presented as fiber material before excels by having, apart from the reinforcing function, also an antibacterial effect.

By mixing the particles, such as magnesium, magnesium-calcium-zinc (MgCaZn), iron, barium, strontium, calcium, hydroxyapatite, alpha- and/or beta-tricalcium phosphate and lime, with the appropriate fibers a time zone/a time development can be regulated such that the degradation behavior of the resorbable material can be accelerated or, as required, can also be decelerated.

In accordance with the invention, it is equally possible to make surface modifications to improve the antibacterial effect of the implant and to optimize the ingrowth behavior. Accordingly, preferably the components magnesium, polyethylene, polypropylene, polyetheretherketone, hydroxyapatite, alpha- and/or beta-tricalcium phosphate and lime have to be modified to bring about the desired behavior of the implant in interaction with the fibers.

Hereinafter, the invention will be illustrated in detail by way of figures, where in this context also various example configurations are explained, wherein:

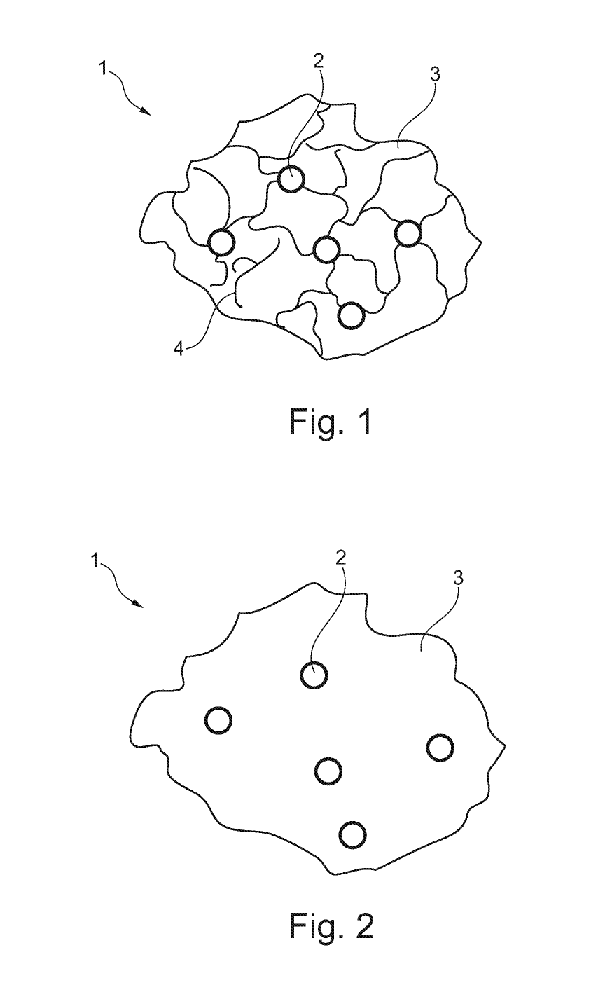

FIG. 1: shows a section across an implant according to the invention in a state shortly after mixing, and

FIG. 2: shows a section according to FIG. 1 in a later state.

The figures are merely schematic and serve exclusively for the comprehension of the invention.

FIG. 1 illustrates a bioresorbable implant 1 for supplementing or replacing hard tissue comprising at least one reinforcing fiber 2. Said fiber 2 in turn includes, according to the invention, at least one of the elements of the group consisting of silk, chitosan, collagen, polycaprolactone, poly(D,L-lactide), poly(lactide-co-glycolide), polyglycolide, polyurethane and polypropylene. The fiber 2 is embedded in a matrix material 3 to form with the latter such implant 1 which exhibits high values of strength both along the longitudinal direction and along the transverse direction. The matrix material 3 is granular or powdery or liquid at the time of being mixed with the fiber 2. This can be seen at the phase boundaries 4 in FIG. 1.

In FIG. 2 a state is shown in which the originally granular composition of the matrix material 3 is completely suspended so that the implant 1 merely includes a homogenous matrix 3 in which reinforcing fibers 2 are disposed. In this state, the implant 1 is preferably adapted to be inserted in a patient.

The design may be flexible. FIGS. 1 and 2 merely illustrate the material composition while the superior implant 1 has to be designed such that it will complementarily supplement the hard tissue to be supported.

The bioresorption of the implant 1 is increased by the fact that in the state shown in FIG. 2 no more phase boundaries 4 will occur.

1. A bioresorbable implant for supplementing or replacing hard tissue and/or soft tissue, comprising at least one reinforcing fiber which is made from a first material component and which, after being mixed with a second material component, is embedded in a matrix, wherein the material of the first material component contains at least one of the elements of the group consisting of silk, chitosan, collagen, polycaprolactone, poly(D,L-lactide), poly(lactide-co-glycolide), polyglycolide, polyurethane and polypropylene, characterized in that the second material component is present in granular or powdery form at the point in time at which the material component is mixed with the at least one reinforcing fiber wherein the entirety of fibers contains at least two of the elements of the group consisting of silk, chitosan, collagen, polycaprolactone, poly(D,L-lactide), poly(lactide-co-glycolide), polyglycolide, polyurethane and polypropylene.

2. The bioresorbable implant according to claim 1, characterized in that the matrix is made from the second material component and at least one further material component.

3. The bioresorbable implant according to claim 1, characterized in that the second material component comprises ceramic phosphate-based components.

4. (canceled)

5. The bioresorbable implant according to claim 1, characterized in that the fiber includes at least one elevation and/or recess to increase an interacting surface between the matrix and the fiber.

6. The bioresorbable implant according to claim 1, characterized in that plural solid particles which enable a resorption time to be controlled are arranged in the matrix, wherein the solid particles include mass percentage of from 5% to 25% as measured by the mass of the bioresorbable implant.

7. The bioresorbable implant according to claim 1, characterized in that mixing of the individual material components is enabled by generative, subtractive and 3D-shaping fabrication.

8. The bioresorbable implant according to claim 1, characterized in that the particles of the second material component which are granular or powdery at the time of mixing adopt an integral and merging shape after a predetermined time window.

9. The bioresorbable implant according to claim 1, characterized in that the particles of the second material component which are granular or powdery at the time of mixing contain at least one of the elements of the group consisting of magnesium, calcium, hydroxyapatite, alpha- and/or beta-tricalcium phosphate and lime to specifically influence the degradation behavior of the bioresorbable implant.

10. A method for producing a bioresorbable implant according to claim 1, comprising the steps of:
providing the at least one fiber from the first material component;
providing granular or powdery particles of the second material component;
mixing the first material component with the second material component to obtain the bioresorbable implant;
post processing the bioresorbable implant as to its shape and/or surface texture so that it is configured to be complementary to the hard tissue and/or soft tissue subject to replacement.
